(19) United States
(12) Patent Application Publication
(10) Pub. No.: US 2006/0251542 A1
(43) Pub. Date: Nov. 9, 2006

(54) Method for sterilizing articles using carbonated water

(76) Inventor: Marc Sims, Berkeley, CA (US)

Correspondence Address: Harris Zimmerman;Law Offices of Harris Zimmerman, Suite 710, 1330 Broadway, Oakland, CA, 94612, US

(21) Appl. No.: 11/123,691

(22) Filed: May 5, 2005

(51) Int. Cl. A61L 2/20

(52) U.S. Cl. 422/33

(57) ABSTRACT

A method of sterilizing, disinfecting and/or sanitizing articles such as medical instruments, medical implants, food handling or processing devices, devices used in space exploration, or any other article in which sterility or biological passivity is required. In general terms, the method comprises placing the article in a pressure chamber, filling the chamber with water so as to cause the article to be submerged, sealing the chamber, mixing carbon dioxide in the water, maintaining a greater than atmosphere pressure in the chamber, and controlling the temperature of the water with dissolved carbon dioxide therein.

### CROSS-REFERENCE TO RELATED APPLICATIONS

Not applicable.

### FEDERALLY SPONSORED RESEARCH

Not applicable.

### SEQUENCE LISTING, ETC ON CD

Not applicable.

### BACKGROUND OF THE INVENTION

1. Field of the Invention

This invention relates to the sterilization, disinfection and sanitization of articles such as medical instruments and medical implants by exposing the articles to pressurized water containing dissolved carbon dioxide. More particularly it relates to the method and apparatus for bringing the pressurized water containing dissolved carbon dioxide into contact with the articles for the purpose of inactivating microorganisms, infection agents, enzymes, and other biologically active contaminants on or in the articles. The term “articles” as used herein is not intended to be limited to medical products, but applies to many products, such as those used in the handling of food, or in space exploration, or any type of product in which sterility and/or biological passivity is required.

2. Description of Related Art

As stated above, numerous articles are required to have a high degree of sterility and/or biological passivity so that their intended use will not result in bodily infection, physiological reaction, or contamination to another article, an organism, or the atmosphere.

For example, articles used for medical implants and invasive medical instruments must have a high degree of sterility and biological passivity so as not to cause bodily infection or physiological reaction. Articles used to process foods and pharmaceuticals require varying degrees of microbiological decontamination so as to produce safe products. Articles such as space exploration devices need to be free of terrestrial microorganisms and their artifacts.

The most common way to sterilize articles is by the use of steam. Steam inactivates biologically active microorganisms, protein, carbohydrates, lipids and nucleic acids. However, articles such as medical endoscopes and other medical devices are now frequently made using heat sensitive polymers, optical fibers, microelectronics, etc. Sterilizing steam is used at a temperature above 100° C. and therefore cannot be used to sterilize the heat sensitive articles.

There is a need to have sterilizing and cleaning methods for the heat sensitive articles which do not use a temperature above around 60° C. and which do not destroy the articles. Most low temperature methods involve applying disinfecting or sanitizing chemicals to the articles. However, the chemicals must be thoroughly rinsed from the articles because of their toxicity, reactivity and potential for irritation which would be caused by the chemical residue. For example, medical endoscopes are cleaned with chemicals such as glutaraldehyde, alcohol, peracetic acid and others. As a last step, the chemicals are required to be rinsed off the articles using sterile water. Producing and aseptically storing the required sterile water is a separate expensive task.

More recently, pressurized carbon dioxide has been proposed as the disinfecting and sanitizing chemical to be used in treating heat sensitive articles. Articles are placed in a pressure vessel. The vessel is sealed and then filled with gaseous, liquid or supercritical carbon dioxide. When processing is completed the pressure is released and the carbon dioxide dissipates leaving no harmful residue on the article. Carbon dioxide is non-toxic, non-flammable, inexpensive and essentially chemically inert to article materials of construction. Release of carbon dioxide into the atmosphere is unregulated.

By way of example, U.S. Pat. No. 3,442,660 to Shank, discloses inoculation of meat pieces with spores of Clostridium 3679, sealing the meat in a can, and pressurizing the can with 120 psi carbon dioxide. After several days, the meat was removed and tested for spores. The reduction in the spore count was reported to be about a factor of 10⁶ (Example IV of Shank).

Biological contaminants on the articles may include enzymes. In U.S. Pat. No. 3,442,660, Shank treated meat pieces with enzymes and then exposed the pieces to pressurized carbon dioxide for several hours to obtain meat substantially free of active enzymes. The enzymes used by Shank are proteolytic and would be particularly hazardous contaminants on medical instruments or implants because these enzymes destroy animal and human tissue.

Biological contaminants may also include viruses. Inactivation of viruses by treatment with pressurized carbon dioxide is reported in: U.S. Pat. Nos. 5,667,835; 5,723,012; and 5,887,005.

Methods of disinfecting and sanitizing articles with carbon dioxide inside pressure chambers are described in Ger. Offen. DE 10,107,831A1 (Chem. Abs. 137:184780) and Ger. Offen. DE 19,852,070 (Chem. Abs. 132:330858). Additional methods describing disinfection of articles with carbon dioxide which is augmented with UV, ultrasonic energy or chemical additives including ethylene oxide and ozone are found in U.S. Pat. Nos. 5,213,619; 6,558,622; PCT publication US9916221; WO2005000364; and Appl. DE 200210236791. However, adding reactive chemicals to the carbon dioxide eliminates important advantages of carbon dioxide including its lack of hazardous residue and its chemical inertness.

In U.S. Pat. No. 6,149,864, Experiment 5, the carbon dioxide sterilization of E. coli bacterial cells is described in which a small amount of water is added to the cells in a pressure chamber system. However, the majority of the void volume of the chamber is filled with pressurized carbon dioxide. It is well known that the presence of water enhances the disinfecting and sanitizing action of the pressurized carbon dioxide and is described quantitatively by Taniguchi, et al, in Agric. Biol. Chem., 51(12), 1987, 3425-3426, and by Kamagai et al, in Biosci. Biotech. Biochem. 61(6), 1997, 931-935.

The continuous sterilization or pasteurization of water based pumpable liquids by exposing them to carbon dioxide is also known and is described in: U.S. Pat. Nos. 5,667,835 and 6,331,272 B1 and in U.S. published application 2002/0122860 A1. In the continuous and batch processes which treat an aqueous liquid, the microorganisms are in the liquid, not on a solid article. However, solid articles referred to in the present invention are not liquid, are not pumpable, and remain stationary in the chamber.

In the above examples of treating articles with carbon dioxide, the majority of the void volume of the treating chambers is occupied by pressurized carbon dioxide. Sealing articles in a pressure vessel chamber and charging the chamber so that the void volume is filled with dry or moisturized pressurized carbon dioxide presents hazards and problems. One hazard is that carbon dioxide is a very compressible fluid. If the chamber wall ruptures or the closure fails due to the high pressure, the resulting explosion can be quite destructive. The expanding carbon dioxide can propel vessel fragments at lethally high velocity and force.

In contrast, filling a vessel's void volume with water, even water containing some dissolved carbon dioxide, is inherently much safer because water has little compressibility. A crack in the vessel would result only in the leaking of a small amount of fluid which rapidly drops the fluid pressure to atmospheric. Pressure vessels are routinely pressure tested hydrostatically, and it is well known to be a safe procedure.

Other problems with vessels filled primarily with carbon dioxide, as taught in the prior art, are the time and cost associated with moving, storing and recycling a large volume of the pressurized gas. During startup, carbon dioxide must be pumped into the vessel from storage, and them pumped back to storage at the end of the cycle. This requires time, a large storage volume and a pump or compressor. As the pressure of carbon dioxide in the vessel drops below about 20 bar, recycling all the gas by recompressing it back to the storage pressure becomes more expensive than venting it. That amount that cannot be economically recycled is large.

Simply venting a vessel filled primarily with carbon dioxide to the atmosphere after a process cycle with therefore add to the operating costs. Also, venting the vessel quickly chills the articles due to vaporization and gas expansion and can even leave the depressurized vessel partially filled with dry ice.

The present invention is directed to overcoming one or more of the problems discussed above.

### BRIEF SUMMARY OF THE INVENTION

The present invention provides a method and apparatus for sterilizing, disinfecting, sanitizing and biologically inactivating articles by exposing the articles to pressurized water containing dissolved carbon dioxide. The method includes the steps of sealing the articles inside a pressure vessel chamber via a closure and filling the void volume of the chamber nearly full with water. The chamber is then pressurized with carbon dioxide and the carbon dioxide is induced to dissolve into the water by stirring, agitating or recirculating the water and carbon dioxide using an external or internal carbonating device until a desired concentration of dissolved carbon dioxide is obtained. The temperature of the carbonated water is adjusted to a desired value by heating the chamber or by recirculating the carbonated water through a heating device. The pressure of the chamber is maintained at a desired value during the heating step by a regulating relief valve set to release fluid at the desired pressure. The articles are held at the desired pressure and temperature for a holding time sufficient to obtain the target level of decontamination. During the holding period, the carbonated water can be static, stirred or recirculated by the pump. At the end of the holding period, the pressure is released by venting the chamber through a valve, and the now sterile water may be retained in the chamber for recycle or it may be drained and discarded. The chamber closure is opened and the treated articles are removed.

### BRIEF DESCRIPTION OF THE DRAWINGS

FIG. 1 is a chart illustrating the solubility of carbon dioxide in water as determined by pressure and temperature changes.

FIG. 2 is a diagrammatic representation of apparatus which may be utilized in carrying out the method of this invention.

FIG. 3 is another diagrammatic representation of apparatus utilized in carrying out the specific example of the invention hereinafter set forth.

### DETAILED DESCRIPTION OF THE INVENTION

In practicing the present invention, a disinfecting and sanitizing fluid is prepared by dissolving carbon dioxide into water at an elevated pressure. This fluid will be referred to as supercarbonated water. The supercarbonated water is brought into contact with solid articles, not shown, so as to disinfect and sanitize the surface of the articles including the interior surfaces of pores if the articles contain materials of construction which are porous. Microorganisms and other biological contaminants on the surfaces are inactivated by contact with the supercarbonated water. The articles and supercarbonated water are disposed in a chamber.

It is known that the extent of inactivation of microorganisms suspended in supercarbonated water depends on the type of microorganism, starting concentration of the microorganism in the water, concentration of dissolved carbon dioxide, temperature and treatment time.

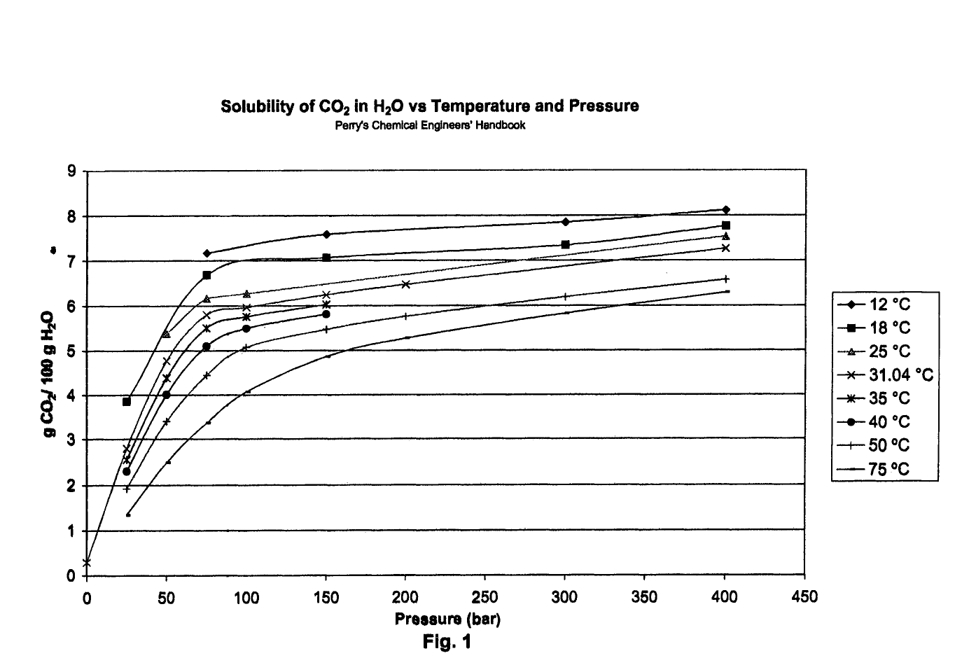

In this invention, supercarbonated water is brought into contact with microorganisms which are not in suspension but which are adhering to the surfaces of the articles. Surprisingly, the kinetics of inactivation of surface microorganisms contacted by the supercarbonated water appear to follow a similar trend. Inactivation rate of surface microorganisms is observed to increase with temperature and concentration of dissolved carbon dioxide. The maximum concentration of dissolved carbon dioxide in water is determined by its saturation solubility at a specified temperature and pressure. The saturation solubility relationship with temperature and pressure is shown in FIG. 1.

For example, at an operating temperature of 50° C. and pressure of 100 bar, FIG. 1 shows that the maximum solubility of carbon dioxide in water (saturation) is 5.1 g carbon dioxide per 100 g water. The carbonation step can be performed at 50° C. and 100 bar until the saturated concentration is achieved. In practice, the time required to reach carbon dioxide saturation can be quite long, depending on the efficiency of the carbonating device. However, it is obviously desirable that the entire process be completed as quickly as possible.

The carbonation step can be accelerated by manipulating the pressure and temperature. The carbonation step can be performed at, for example, 18° C. and 75 bar, and a concentration greater than 5.1 g carbon dioxide per 100 g water or more is easily achieved since the maximum solubility is over 6 g carbon dioxide per 100 g water under these conditions.

Next, the near-saturated supercarbonated water is heated in the chamber receiving the articles, excess carbon dioxide comes out of the solution, and the chamber pressure rises. A pressure regulating valve set to maintain 100 bar, for example, will release excess carbon dioxide. When the temperature of 50° C., for example, is reached, the supercarbonated water will be at its maximum concentration, i.e. saturated with dissolved carbon dioxide. It will have maximum disinfecting and sanitizing power at this example temperature and pressure.

While it is desirable that the concentration of dissolved carbon dioxide be maximized to reduce processing time, it is understood that the method of the invention is still useful for inactivating microorganisms and biologically active contaminants even when the dissolved carbon dioxide concentration is below the saturated value.

Devices for carbonating water and water-based liquids are well known. One general type of device disperses the carbon dioxide into the liquid so that bubbles or droplets are formed. This type of device includes sintered metal spargers, static mixers, packed columns, injectors or agitators which disperse the mixture. Another type of carbonator does not disperse one fluid in another but makes use of a membrane to keep the fluids separate while allowing carbon dioxide to diffuse into the water. The hallmark of membrane gas transfer devices is that a large amount of active interfacial area can be contained in a small volume device leading to higher efficiency. A 10-fold increase in gas transfer efficiency of a hollow fiber membrane device over a conventional packed column was reported by Yand, et al, AlChE J., 32, 1986, 1910-1916. Examples of membrane carbonation are given by Karoor, et al, Ind. Eng. Chem. Res., 32, 1993, 674-684, and in U.S. Pat. No. 6,331,272 B1.

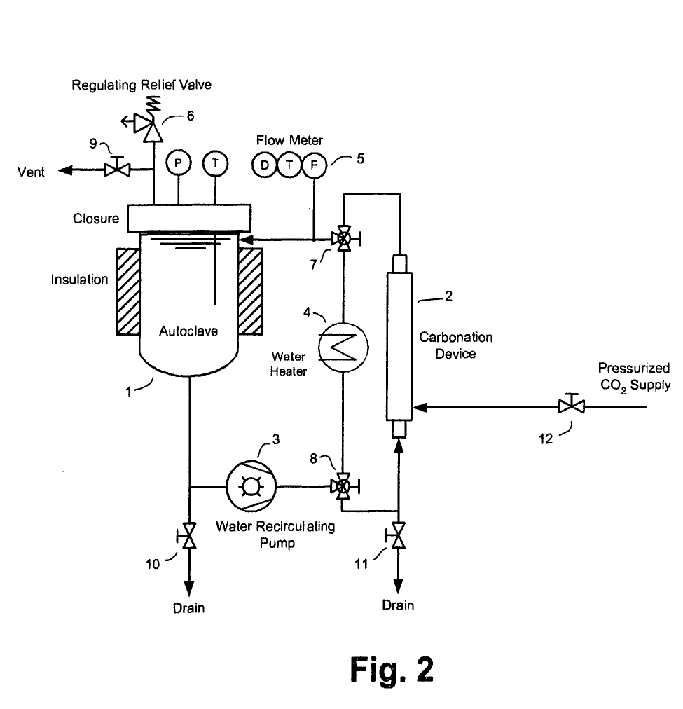

One embodiment of the new invention is shown in FIG. 2. An autoclave 1 with a top closure serves as the pressure vessel chamber for the articles. Ideally the closure will be of the type which opens and closes rapidly. After the articles are placed into the autoclave 1, water is introduced so that nearly all the void volume in the autoclave 1 is filled with water and all the articles are submerged in the water. With the closure sealed shut, carbon dioxide is introduced to the autoclave 1 by opening valve 12. The pressurized carbon dioxide supply can be a refrigerated storage tank with a compressor, or a standard commercial compressed gas cylinder at ambient temperature. The three-way ball valves 7 and 8 are set so all flow from the water recirculating pump 3 is directed into the carbonation device 2 and back out into the autoclave 1.

Water recirculation is begun by turning on the pump 3. The pump speed can be varied to obtain the desired flow rate. An optional flow meter 5 would indicate the water flow rate. Another option is that flow meter be a coriolis type which also measures and indicates density and temperature. The density of carbonated water at a particular temperature and pressure correlates with dissolved carbon dioxide concentration. The flow meter 5 would then usefully indicate dissolved carbon dioxide concentration on line.

The regulating relief valve 6 is set to a desired release pressure which is the desired maximum operating pressure. The water heater 4 will heat the water and maintain it at a constant desired temperature as indicated by the temperature sensor. Recirculation of the water is continued for the desired time at the desired pressure as indicated by the pressure sensor.

At the end of the recirculation and holding period, the pressure is released via valve 9, or drain valve 10, or both. The now sterile water can be drained from the autoclave and discarded or it can be retained for reuse on the next batch of articles to be treated.

It is understood that the articles can be pre-cleaned in the conventional way prior to the method of the new invention. For example, it is recommended that endoscopes are to be scrubbed and flushed with an enzymatic detergent solution which is then to be rinsed off with clean water. It is also recommended that endoscopes be rinsed with sterile water after a treatment step with a sterilizing chemical solution.

The pre-cleaning steps can be done in a separate vessel or could advantageously be done in autoclave 1. Rinsing and draining the cleaning solution from the autoclave 1 could be followed by the method of the new invention. At the end of the new method, the articles remain submerged in sterile water which was produced in the method. Thus the new method eliminates the need and expense of having a separate system for producing sterilized rinse water and storing it aseptically.

Dissolving carbon dioxide in water produces carbonic acid. At the pressures contemplated in the practice of the method described herein, the pH of the carbonated water can be as low as 2.8. Some articles may be made of materials which are sensitive to this acidity. Fortunately, buffer can be added to the water to prevent the pH from dropping too low. Holmes, et al, Langmuir, 14, 1998, 6371-6376, demonstrated a number of effective buffers for the carbon dioxide in water system. For example, a concentration of 0.1 molar sodium bicarbonate in water prevented the pH from dropping below 5.1 when the water was saturated with dissolved carbon dioxide at 450 bar and 20° C.

The presence of buffers is not observed to interfere with the inactivation of microorganisms by carbon dioxide. For example, 0.1 molar concentration of buffer did not prevent the inactivation of Lactobacillus plantarum suspended in water by treatment with pressurized carbon dioxide as reported by Hong, et al, J. Food Sci., 64(4), 1999, 728-733.

If a buffer is added to the water in the new method it should itself be physiologically benign. Sodium bicarbonate is such a benign buffer. It is, for example, a natural component of mammalian blood in its dissolved form of sodium and bicarbonate ions.

In some cases, a contaminant of an article may be resistant to inactivation by treatment with supercarbonated water alone. In such cases, adding a chemical to the water which provides additional decontamination power may be desirable, provided the additive is chemically mild toward the material of construction of the article, and provided the additive does not leave a hazardous residue on the article.

Percarbonic acid, a mild oxidizer and sanitizer, is an additive with these attributes. Its degree of reactivity in the supercarbonated water depends on its concentration, which can be adjusted accordingly. Upon depressurization, percarbonic acid in the system decomposes to harmless carbon dioxide, water and oxygen.

Percarbonic acid is conveniently prepared in situ by adding an appropriate amount of hydrogen peroxide to the water in the present system. Upon carbonation of the system water, the formed carbonic acid spontaneously combines with hydrogen peroxide to form percarbonic acid.

It will be understood by one skilled in the art that the operating conditions of the new method can be varied so as to optimize it, and that the operations of components can be automated using programmable logic controllers, computers, actuators, and the like.

The maximum temperature of the method will be determined by the temperature sensitivity of the articles. Generally a higher temperature is preferred because it accelerates the disinfecting and sanitizing kinetics. The preferred range is 20° C. to 70° C.

The maximum operating pressure is that which is high enough to obtain a fast processing cycle but not so high as to require an expensive heavy construction for the autoclave. It is understood that a balance of conditions can be found such that cycle time, operating cost and equipment cost are optimized. The preferred range of operating pressure is 5 bar to 200 bar.

In the new method, the time of holding at a particular temperature and pressure is set by the susceptibility of the microorganisms and biological contamination to inactivation at the operating conditions. In general, the time is chosen to just obtain the level of sanitization, disinfection and decontamination desired for the articles. The preferred range of the holding time is 0.1 hours to 10 hours.

The preferred concentration of dissolved carbon dioxide is the saturated concentration at the operating temperature and pressure.

The preferred level of filling of the chamber with supercarbonated water is such that the majority of the void volume is filled by it and all the articles are submerged in it.

### EXAMPLE

Porous sintered 316 stainless steel circular discs with nominal pore size of 10 micrometers were used as test articles. The discs were 2.68 cm diameter×1.65 mm thick. In addition, disc “sandwiches” were prepared by sticking two of the discs together after applying a circular bead of hot melt polyolefin glue around the periphery of a disc near its outer edge. While the glue was still melted, two discs were pressed together leaving a cavity between the two. A “sandwich” was clamped with small office supply bulldog clips to prevent the discs from separating.

Two of the single discs and two “sandwiches” were inoculated with B. subtilis spores. An inoculated pair of articles consisting of one disc and one “sandwich were treated by the method, and the other inoculated pair were used as controls.

B. subtilis microorganism (ATCC 6633) was purchased from Cryocults of Newbury Park, Calif. Cultures were grown in standard HBI broth for 36 hours at 36° C. The broth was then centrifuged and the pellets resuspended in Msgg (poor nutrient) broth and kept at room temperature for 24 hours. This broth was concentrated by centrifugation and the pellets resuspended in Butterfield's phosphate buffer producing a cloudy spore inoculum of about 10⁹ CFU/cc. The inoculum was used immediately.

Both the single discs and “sandwiches” were soaked in the inoculum for 5 minutes, drained of excess liquid and allowed to air dry before use.

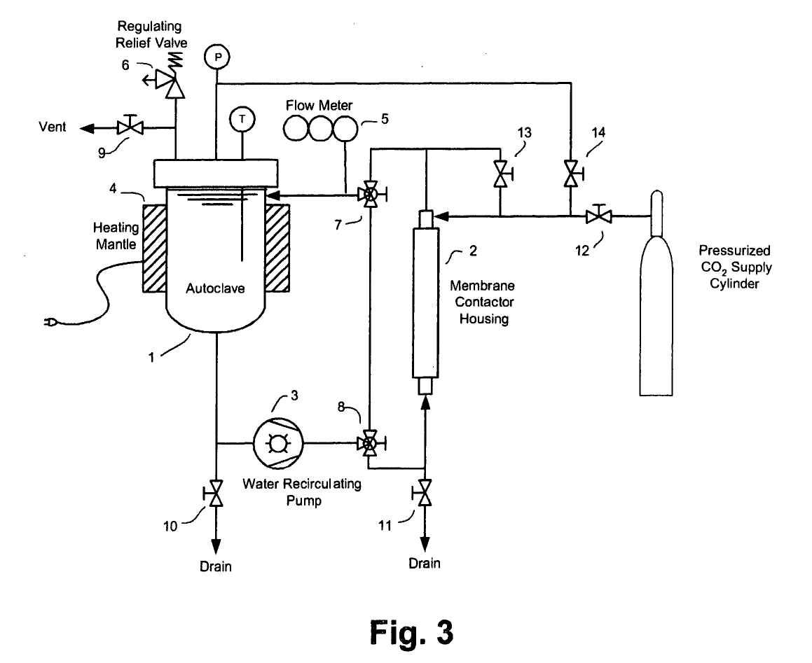

The apparatus used in the example is shown in FIG. 3. The autoclave 1 in the example apparatus is a 300 cc Autoclave Engineers 316 stainless steel bolted closure model BC0030SS05 with an electric heating mantle 4 powered by a variable voltage transformer. A disc and a “sandwich” were placed on a stainless steel wire platform resting in the bottom of the autoclave 1. The autoclave 1 was filled to the top with distilled water and the closure was bolted closed.

Three-way ball valves 7 and 8 were set so that all water flow from the recirculating pump 3 was directed into the membrane contractor 2 and back out into the autoclave 1. The pump 3 is a Micropump model 67-L21311 magnetic drive gear pump with variable speed drive. The membrane cartridge is a Celgard model Liqui-Cel Extra Flow 2.5×8, with polypropylene microporous hollow fibers, having an effective surface area of 1.4 m². The cartridge was sealed inside a stainless steel cylinder housing specially constructed to operate at a system pressure of 200 bar. In the cartridge, carbon dioxide was in the fiber lumens and water flowed on the shell side.

The pressure equalizing valves 13 and 14 were opened. The carbon dioxide supply cylinder was warmed to about 10° C. above room temperature so that the vapor pressure was 75 bar. The carbon dioxide valve 12 was slowly opened to allow the entire system pressure to rise uniformly to 75 bar. Valve 13 was then shut. Valve 14 was left open so hat the water side of the membrane communicated with the carbon dioxide side and would prevent a destructive transmembrane differential pressure if the pressure on either side of the membrane were to change abruptly due to a system upset.

Water circulation through the membrane contactor was begun by turning on the pump 3 and adjusting the flow rate to 100 g/min as indicated by the Micromotion coriolis flow meter 5, model CMF010P323DRAUEZZZ. This flow rate was maintained constant throughout the example.

After 4 minutes of water circulation through the membrane contactor, the water density indicated by the flow meter 5 reached a maximum of 1.0164 g/cc at 25.8° C. and remained constant, indicating that near saturation with dissolved carbon dioxide was achieved.

Valves 7 and 8 were then set so that water bypassed the membrane contactor. Valves 12 and 14 were shut off to isolate the autoclave. Heating of the circulating water was begun by turning on the electrical heating mantle 4. Temperature of the water reached 47° C. in 50 minutes after the start of water recirculation. The mantle voltage was adjusted to maintain the temperature constant at 47° C.

During the heat up period, the pressure in the autoclave 1 rose until carbon dioxide began venting from the regulating relief valve 6 set to release at 140 bar. The pressure reached 140 bar 40 minutes after the start of water circulation.

Water recirculation was continued at 47° C. and 140 bar until 60 minutes after starting circulation. The pressure was then slowly released by opening valve 9. Recirculation of water at 47° C. was continued during the depressurization. Pressure in the autoclave 1 reached atmospheric when the total elapsed time form start up was 80 minutes. Then the mantle 4 and recirculating pump 3 were turned off. The autoclave closure was opened and removed.

The water in the chamber was still warm because the thermodynamic cooling effect of releasing dissolved carbon dioxide is low and creates only a 1°-2° C. temperature drop of the water.

The disc and sandwich treated in the example were removed with forceps from the autoclave water. Each of the treated articles and control articles were individually soaked in a 150 cc portion of sterile phosphate buffer for 10 minutes. Each phosphate buffer portion plus a sample of water taken from the autoclave were plated in petri dishes using the standard spread plate technique. Separately, the discs and “sandwiches” were laid on top of PDA medium with 5 cc of sterile phosphate buffer solution in petri dishes. All plates and plated articles were incubated at 38° C. for 48 hours and observed every 12 hours for signs of growth.

TABLE 1
Growth Tests

| | | | Total |
|---|---|---|---|
| Sample | Description | Growth | Kill |
| Dip1 | Processed Disc | | ✓ |
| Dip2 | Control Disc | ✓ | |
| San1 | Processed Sandwiched Discs | | ✓ |
| San2 | Control Sandwiched Discs | ✓ | |
| Dip1W | Wash Water From Processed Disc | | ✓ |
| Dip2W | Wash Water From Control Disc | ✓ | |
| San1W | Wash Water From Processed Sandwich Discs | | ✓ |
| San2W | Wash Water From Control Sandwich Discs | ✓ | |
| WA | Processed Water From Autoclave | | ✓ |

Observation of any significant growth is represented by a check mark in the Growth column. Observation of no significant growth is represented by a check mark in the Total Kill column. The results show that a complete kill of the B. subtilis was achieved in the example.

While the invention has been described with reference to a specific embodiment for purposes of example, many modifications and variations are possible and it is not intended to limit the invention except as defined in the following claims.

1. A method of sterilizing, disinfecting, sanitizing and/or reducing the biological activity of an article, including the steps of:
placing said article in a pressure chamber;
introducing water in said chamber so as to submerge at least the contaminated portion of said article;
sealing said chamber;
introducing compressed carbon dioxide into said sealed chamber;
causing the carbon dioxide to dissolve in the water so that at least said contaminated portion of said article is submerged in and exposed to carbonated water;
maintaining said article in said chamber and exposed to said carbonated water for a period of time so that a desired level of sterility and/or level of disinfection, sanitization, or biological inactivity is obtained.

2. The method of claim 1 in which water is introduced into said chamber until the greater portion of the void volume of said chamber is filled with water.

3. The method of claim 1 in which said carbon dioxide is induced to dissolve in the water.

4. The method of claim 3 in which a carbonation device is used to induce said carbon dioxide to dissolve in said water, the mixing of carbon dioxide and water being external to said chamber.

5. The method of claim 3 in which the carbon dioxide and water is mixed inside said chamber.

6. The method of claim 4 in which the carbonation device is a membrane contactor in which said water is recirculated through said chamber so as to cause carbon dioxide to dissolve in the water by diffusion at the membrane interface.

7. The method of claim 1 wherein a buffer is added to the water in said chamber in an amount to keep the pH of said water at a level greater than the pH of pure carbonic acid.

8. The method of claim 7 in which said buffer is sodium bicarbonate.

9. The method of claim 8 in which said buffer is at a concentration substantially in the range of 0.01 molar to 1.0 molar.

10. The method of claim 1 in which percarbonic acid is provided in the water of said chamber.

11. The method of claim 10 in which said percarbonic acid is prepared in the chamber by adding hydrogen peroxide to the water.

12. The method of claim 1 in which the temperature of the water is monitored and controlled.

13. The method of claim 12 in which the temperature of the water is maintained in a range of substantially 20° C. to 70° C.

14. The method of claim 1 in which the pressure within said chamber is monitored and controlled.

15. The method of claim 14 in which the pressure is maintained in a range of substantially 5 bar to 200 bar.

16. The method of claim 1 in which said article is precleaned by adding a cleaning solution to the chamber and flushing the same from the chamber before introducing water to the chamber.
